(19) United States
(12) Patent Application Publication
(10) Pub. No.: US 2012/0123559 A1
(43) Pub. Date: May 17, 2012

(54) PUMP COMPRISING A MOVING WALL AND USE OF A PUMP OF THIS TYPE

(75) Inventors: Lüder MOSLER, Duderstadt (DE); Martin HILLMANN, Duderstadt (DE); Ralf CARSTENS, Gottingen (DE)

(73) Assignee: OTTO BOCK HEALTHCARE GMBH, Duderstadt (DE)

(21) Appl. No.: 13/296,816

(22) Filed: Nov. 15, 2011

Related U.S. Application Data
(63) Continuation of application No. 11/572,863, filed on Jan. 29, 2007, now abandoned.

(30) Foreign Application Priority Data
Jul. 28, 2004 (DE) 102004036669.1

(51) Int. Cl. A61F 2/80

(52) U.S. Cl. 623/34

(57) ABSTRACT

The invention relates to a pump comprising a wall, which forms an enclosed fluid volumetric space and which can be moved by a first force in a direction resulting in a decrease in volume and, after a preceding decrease in volumetric space, can be moved by a second force in a direction resulting in an increase in volume. The pump also comprises an inlet valve, which communicates with an inlet valve, and comprises an outlet valve in an outlet line of the fluid volumetric space. The inventive pump has a compact design due to the fact that it is provided in the form of a vacuum pump whose volumetric space can be decreased by the external first force acting against an elastically deformable material whose restoring force, after the external force ceases, generates the second force acting counter to the generated vacuum.

### CROSS-REFERENCE TO RELATED APPLICATION

This application is a continuation of application Ser. No. 11/572,863, filed Jan. 29, 2007, pending, which is a 371 of PCT/DE2005/001124 filed on Jun. 24, 2005 and published on Feb. 9, 2006 as WO 2006/012820, which claims priority to German Patent Application No. 10 2004 036 669.1, filed 28 Jul. 2004, the disclosure of each of which is incorporated, in its entirety, by reference.

### TECHNICAL FIELD

The invention relates to a pump comprising a wall which forms a closed-off fluid volume and which can be moved by means of a first external force in the direction of a volume reduction and by means of a second force, after a preceding volume reduction, in the direction of a volume increase, and with an inlet valve which communicates with an inlet port and with an outlet valve in an outlet line in the fluid volume.

The invention relates, furthermore, to a use of a pump of this type.

### BACKGROUND OF THE INVENTION

Pumps of this type are known, for example, as hose pumps, in which the fluid is expressed from a fluid-filled hose by means of pressure rollers pressing the hose together and moved in a longitudinal direction of the hose. As a result of the movement of the roller, the upstream end of the hose is filled with fluid again when this end is connected to a fluid supply. The propulsive force for moving the rollers is generated by a motor which may be designed, for example, as an electric or hydraulic motor. Pumps of this type are used for conveying a volume in the pressure direction.

Another kind of pump of the type initially mentioned is diaphragm pumps, in which the fluid volume is reduced by the diaphragm and moved back and forth by means of a connecting rod and is subsequently increased again. The connecting rod thus transmits both the first force for the volume reduction and the second force for the volume increase.

In many instances, it is merely necessary to call up a pumping power only in specific operating states when movements, the force flux of which can be used for actuating a pump, take place in a device. Thus, it is known, for example, to evacuate the interspace between a patient's amputation stump and an airtight liner arranged above it, in order, by means of the vacuum formed, to ensure a firm fit of the liner connected to a prosthesis. For this purpose, a piston pump is used, which, when the patient treads on the ground by means of the prosthesis, exerts an evacuation stroke and is returned by means of a return spring. Pumps of this type are relatively bulky particularly because of the return mechanism required.

The object on which the invention is based is, therefore, to design a pump of the type initially mentioned such that it can be implemented in a small space.

### SUMMARY OF THE INVENTION

To achieve this object, according to the invention, a pump of the type initially mentioned is characterized in that it is designed as a vacuum pump, the volume of which can be reduced by means of the external first force against an elastically deformable material, the return force of which forms, after the termination of the external force action, the second force acting counter to the generated vacuum.

In the pump according to the invention, the working stroke by which the fluid, in particular air, is sucked away from a closed-off volume, is brought about by the return force of the elastically deformable material. Previous deformation for reducing the volume of the fluid material takes place by means of a first force acting externally. The pump according to the invention thus makes it possible to have a very uncomplicated and small-volume design, by means of which a low to medium vacuum can be generated.

In a first preferred embodiment of the invention, the wall has two rigid walls lying opposite one another, the elastically deformable material being arranged in the interspace formed by the walls. In this case, the elastically deformable material may be formed by a sealing insert running around the edge and delimiting the fluid volume. This embodiment affords the advantage that the external force can act directly on one of the rigid walls.

In another embodiment likewise having advantages, the wall is designed flexibly, the elastically deformable material bearing, preferably over a large area, against the flexible wall, In this case, a high return force sufficient for many applications can be generated by means of a relatively thin material layer.

The elastic material may be an open-pored foam which is arranged within the fluid volume and which exerts the return force after a volume reduction has been carried out. The fluid, which is preferably air, accordingly flows through the foam. In this embodiment, the generation of the return force does not take up any additional space at all, since the fluid volume itself is utilized for this purpose. In this case, it is expedient if the foam completely fills the fluid volume, with the exception of residual volumes as a consequence of construction. An alternative elastic material which is capable of throughflow and which can be used for the invention is a wide-mesh knitted fabric.

The pump according to the invention is implemented in a simple way if the elastic material is surrounded on all sides by the flexible wall. It is also possible, however, for the wall to be partially of rigid design and for a part of the wall such as is required for the volume reduction to be made flexible.

The pump according to the invention can preferably be produced with a preferred large-area extent and with a thickness which is small, as compared with this, and can therefore in many instances be integrated into the structure of a device without difficulty.

To press together the fluid volume, at least one pressure element bearing against the flexible wall over a large area is provided. In particular, the fluid volume with a flexible wall may be arranged between two large-area pressure elements.

The valves may be arranged on the corresponding narrow sides of the flexible wall, but are preferably also arranged in recesses of one of the pressure elements or of both pressure elements, with the result that flexing actions of the flexible wall are reduced.

The pump according to the invention can advantageously be integrated in the force flux of a system in which forces arise which are utilized to exert one of the two forces. The pump according to the invention is suitable particularly as a vacuum pump.

In a special application, the pump constitutes part of a prosthesis for a lower extremity. Preferably, in this case, the force occurring due to body weight when a patient treads on the ground is utilized as the first force. The pump may be employed, in particular, for the vacuum assistance of a suction well of the prosthesis, in particular for evacuating the interspace between a liner and the prosthesis shank. A preferred place of use for the pump according to the invention is an artificial foot which makes it possible particularly effectively to have the large-area design of the pump perpendicularly to the force flux occurring during load caused by the body weight.

### BRIEF DESCRIPTION OF THE DRAWINGS

The invention will be explained in more detail below by means of exemplary embodiments illustrated in the drawing in which:

FIG. 1 shows a diagrammatic illustration of a pump in the initial state according to a first embodiment;

FIG. 2 shows the pump according to FIG. 1 in the compressed state;

FIG. 3 shows a pump in an initial state according to a second embodiment;

FIG. 4 shows a pump in a third embodiment which is integrated into an artificial foot;

FIG. 5 shows an arrangement of an elastically deformable material consisting of two layers with profilings which point toward one another and in the nonloaded state form a fluid space;

FIG. 6 shows the arrangement according to FIG. 5 in a loaded state in which the compressed material of the layer completely fills the fluid space;

FIG. 7 shows an embodiment of a pump according to the invention with two rigid plates lying opposite one another and with an insert running around at the edge and consisting of an elastically deformable material, in the nonloaded state;

FIG. 8 shows the arrangement according to FIG. 7 in the loaded state.

### DETAILED DESCRIPTION

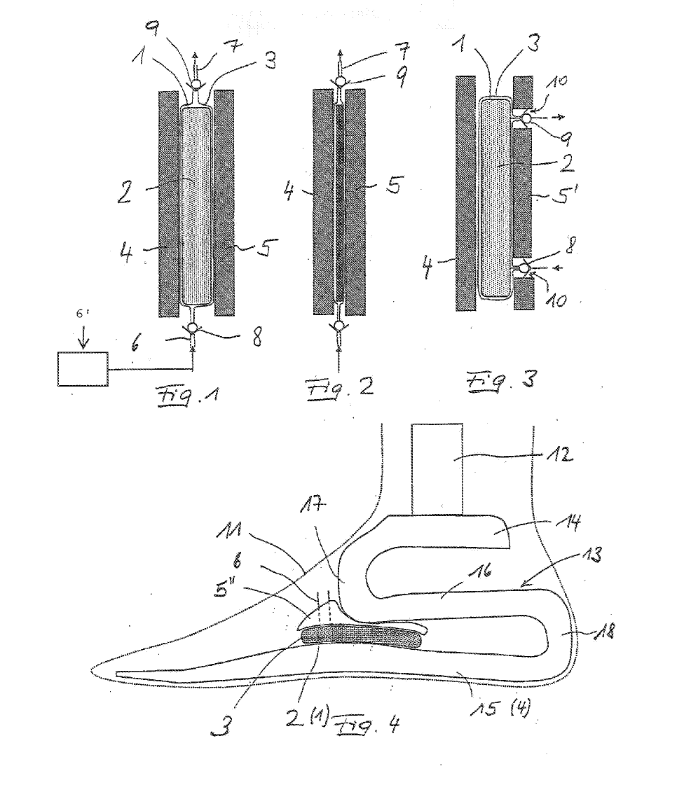

The pump illustrated in FIG. 1 has a closed fluid volume 1 which is filled virtually completely with an elastic material in the form of an open-pored elastic foam 2. The foam 2 has a substantially large-area extent with a small width. It is surrounded on all sides by a flexible wall 3 which thus has four narrow sides and two large-area sides with large dimensioning, as compared with these. Two large-area pressure elements 4, 5 bear against the large-area sides.

The flexible wall 3 has on opposite narrow sides a tubular or hose-shaped feed 6 and a tubular or hose-shaped outlet 7. Both the feed 6 and the outlet 7 are provided in each case with a nonreturn valve 8, 9. The feed 6 is in fluid communication with a closed-off volume 6′.

For the pumping operation, the pressure elements 4, 5 are moved relatively toward one another, as illustrated in FIG. 2. The fluid volume 1 together with the foam 2 contained in it is thereby pressed together, with the result that fluid, preferably air, escapes via the nonreturn valve 9 and the outlet 7. When the pressure force of the pressure elements 4, 5 ceases, the elastic return force of the foam 2 ensures that the fluid volume 1 returns to the initial state of FIG. 1, fluid being sucked into the fluid volume 1 via the inlet 6 and the nonreturn valve 8.

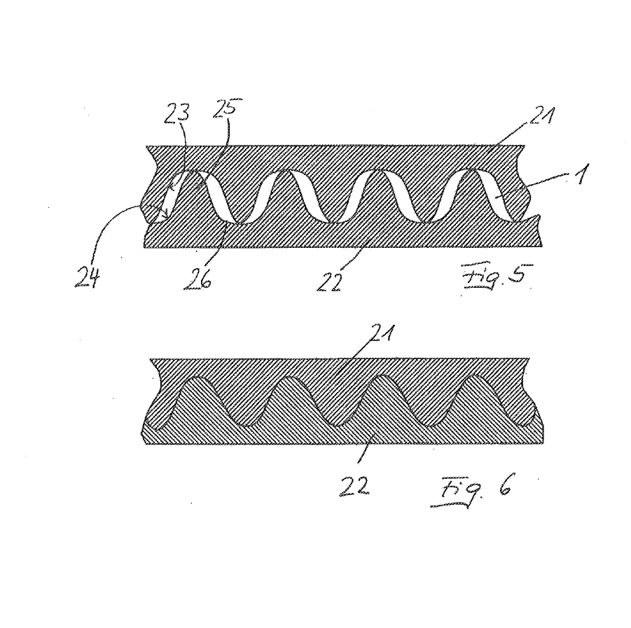

In the modification of the pump, as illustrated in FIG. 3, the pressure plate 5′ is provided with recesses 10 in which the nonreturn valves 8, 9 are arranged, The flexing actions of the flexible wall 3 in the region of the nonreturn valves 8, 9 are thereby markedly reduced.

FIG. 4 shows an example of the use of a pump of the type described in FIGS. 1 to 3. The pump is in this case integrated into an artificial foot 11, the functional set-up of which is connected to a lower-leg tube 12 of a lower-leg prosthesis. The functional part of the artificial foot consists of an S-shaped spring insert 13, the free ends of which form an upper junction limb 14 and a lower sole limb 15. Between these is located an essentially horizontal intermediate piece 16 which is connected to the junction limb 14 and to the sole limb 15 in each case by means of a curved transition piece 17, 18 such that the essentially horizontal intermediate piece 16 can spring in relation to the sole limb 15 of the insert 13 under the action of a weight. Under the action of the weight of the prosthesis wearer when the latter puts his foot onto the ground, therefore, the distance between the intermediate piece 16 and the sole limb 15 is reduced. This distance reduction is utilized for the pump employed according to the invention, in that the intermediate piece 16 is connected to a pressure element 5″. The pressure element 5″, of large area per se, is adapted in its form to the form of the intermediate piece 16 and of the curved transition 17, in order to make it easier to position the pressure element 5″. The fluid volume 1 filled by the foam 2 is located, within the flexible wall 3 surrounding the foam 2 on all sides, between the pressure element 5″ and the sole limb 15 functioning here as a counterpressure element 4. One of the connections 6, 7 is indicated merely diagrammatically, said connection extending through a recess 10 of the pressure element 5″ and being connectable, for example as an inlet 6, via a hose line to the interspace between an amputation stump and a liner surrounding the amputation stump, in order to evacuate this interspace.

The functioning of the pump inserted into the foot 11 corresponds fully to the functioning explained with reference to FIGS. 1 to 3. The load on the artificial foot 11 due to the body weight acts as a pressure force for the pressure element 5″, with the result that the intermediate piece 16 is pressed downward in the direction of the sole limb 15 and thus presses together the fluid volume 1 and the foam 2. Air thereby escapes from the fluid volume 1. When the artificial foot 11 is relieved, at the latest when the foot 11 is lifted off from the ground for the next step, the foam 2 causes the pump to return to the initial position illustrated in FIG. 4. In this case, air is sucked via the inlet 6 into the fluid volume 1 out of the interspace between the amputation stump and the surrounding liner, that is to say a desired vacuum which stabilizes the fit of the liner on the amputation stump is generated in the interspace.

It can be seen that an artificial foot 11 is suitable for integrating the pump according to FIG. 4, because it is easily possible to have a large-area design of the fluid volume 1 and of the foam 2 perpendicularly to the (vertical) force flux because the anatomy of the foot 11 likewise tends to extend over a large area. It is nevertheless also possible, of course, to arrange a corresponding pump in other prosthesis parts which execute a relative movement with respect to one another, for example in a knee joint.

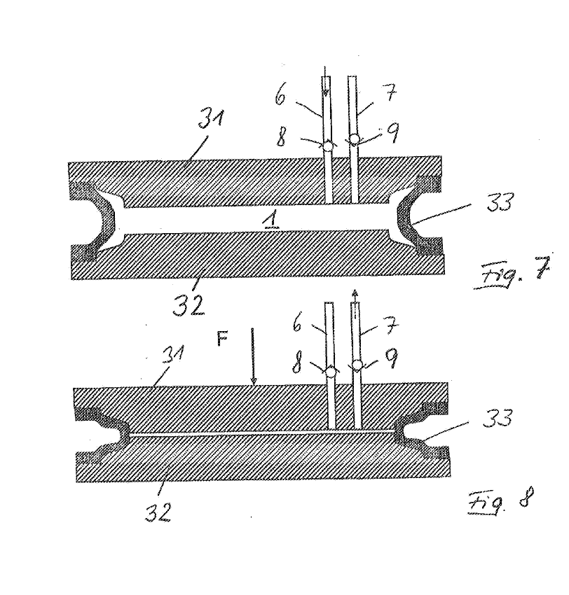

FIGS. 5 and 6 show an exemplary embodiment in which the elastically deformable material is formed by two layers 21, 22 which point toward one another in each case with a profiled surface 23, 24. The profiled surfaces are in this case formed by projections 25, the width of which is smaller than a recess 26 between the projections 25. The tips 25 of the layer 21 accordingly project into the recesses 26 of the layer 22, and vice versa, thus giving rise to the spaces which form the fluid space 1 and which are not filled by the elastically deformable material.

If, then, a pressure causing compression is exerted onto layers 21, 22, the tips 25 are compressed in the height direction and the material is deflected into width, so that the spaces between the tips 25 and the recesses 26 are filled, ideally completely, as illustrated in FIG. 6. When the external force pressing the layers 21, 22 against one another ceases, the initial situation of the layers 21, 22, as is illustrated in FIG. 5, is established. The return causes fluid, in particular air, to be sucked in from a vacuum space connected to the fluid space 1.

The layers 21, 22 may be formed from a plastic having stability such that the layers 21, 22 at the same time form the wall 3. Alternatively, however, the layers 21, 22 may also cooperate with pressure plates 4, 5, such as are illustrated in the embodiments according to FIGS. 1 to 3.

In the exemplary embodiment illustrated in FIGS. 7 and 8, the fluid space is delimited by two rigid walls 31, 32 parallel to one another and by an insert 33 which runs around and connects the edges of the rigid walls 31, 32 sealingly to one another and which consists of an elastically deformable material. The insert 33 is in this case a channel-like profile consisting of a stable fluidtight material which is connected, fluidtight, to the walls 31, 32 by adhesive bonding or the like.

By means of an external first force F, the rigid walls 31, 32 are pressed against one another, as indicated in FIG. 8. The fluid space 1 is thereby reduced to a maximum, with the result that the fluid contained in the fluid space 1 escapes through the outlet 7. When the external force F ceases, the state illustrated in FIG. 7 is reestablished due to the return force of the insert 33, with the result that fluid is sucked into the fluid space 1 via the feed 6.

It can be seen that the exemplary embodiments illustrated in FIGS. 5 to 8 can be employed in the same way as the exemplary embodiments according to FIGS. 1 to 3, that is to say, in particular, also in an artificial foot or the like,

The pumps according to the invention may be used, in particular, in prosthesis parts also for other purposes, for example as a hydraulic pump for the control of dynamic functions, for example for the control of hydraulic damping cylinders or for the movement of structural elements of the prosthesis, for example from an uncoupled to a coupled state, in order to carry out dynamic adaption to the situation of use.

We claim:

1. A prosthetic device for attachment to a lower extremity comprising:
at least one pressure element;
a pistonless vacuum pump comprising an elastically deformable material and a wall forming a closed-off fluid volume adjacent the at least one pressure element, the wall being flexible or having flexible portions so that upon application of a first force exerted by the at least one pressure element the wall flexes, the elastically deformable material compresses, and a volume of the closed-off fluid volume reduces, wherein expansion of the elastically deformable material creates a second force directed to move away the pressure element and create a volume increase in the closed-off fluid volume;
an outlet port in fluid communication with the closed-off fluid volume, which evacuates fluid from the closed-off fluid volume upon the application of the first force; and
an inlet port in fluid communication with the closed-off fluid volume and a vacuum space of the prosthetic device, which draws fluid into the closed-off fluid volume upon application of the second force to create a vacuum condition in the vacuum space.

2. The prosthetic device as claimed in claim 1, wherein the at least one pressure element comprises two spaced rigid walls, and wherein the elastically deformable material is arranged in a space between the two rigid walls.

3. The prosthetic device as claimed in claim 2, wherein each rigid wall comprises an inner edge on a side toward the space, and wherein the elastically deformable material comprises a sealing insert positioned at the inner edges to the fluid volume.

4. The prosthetic device as claimed in claim 2, wherein the space between the rigid walls is reduced upon application of the first force.

5. The prosthetic device as claimed in claim 1, wherein the elastically deformable material comprises two layers that contact one another, each layer including a surface profile that is complementary of the profile of the other layer, with each layer positioned so that each profile points toward the other and forms spaces therebetween, and wherein the spaces are reducible by means of the first force.

6. The prosthetic device as claimed in claim 1, further comprising:
a non-return inlet valve in fluid communication with the inlet port; and
a non-return outlet valve in fluid communication with the outlet port.

7. The prosthetic device as claimed in claim 6, wherein the inlet and outlet valves are each positioned on a side of the closed-off fluid volume which corresponds to a thickness of the pump.

8. The prosthetic device as claimed in claim 6, wherein the closed-off fluid volume comprises opposing elastically deformable walls and the inlet and outlet valves are each arranged on opposing narrow sides of the wall.

9. The prosthetic device as claimed in claim 6, wherein the inlet and outlet valves are each arranged in a recess of one of the at least one pressure element.

10. The prosthetic device as claimed in claim 1 wherein the first force is caused by the application of body weight to the prosthetic device component.

11. The prosthetic device as claimed in claim 10, wherein the prosthetic device comprises a prosthetic socket into which a residual limb is adapted to be received and the vacuum space is defined between the prosthesis socket and the residual limb.

12. The prosthetic device as claimed in claim 11, wherein the vacuum pump and pressure element are positioned between plates of a prosthetic foot and the first force is generated by the prosthetic foot contacting the at least one pressure element during movement.

13. A prosthetic device, comprising:
a pistonless vacuum pump, comprising:
a wall configured to form a closed-off fluid volume, the wall being flexible or having flexible portions movable by means of a first force which produces a volume reduction of the fluid volume and, after a preceding volume reduction, the wall movable by means of a second force which produces a volume increase in the closed-off fluid volume;
an inlet port in fluid communication with the closed-off fluid volume, the inlet port including a non-return valve configured to be connected in fluid communication to a vacuum space of the prosthetic device;
an outlet port in fluid communication with the closed-off fluid volume, the outlet port including a non-return valve configured to exhaust external to the fluid volume; and
wherein the wall comprises an elastically deformable material that is compressed by the first force and whose expansion, after termination of the first force, produces the second force;
wherein the first force exhausts fluid from the closed-off fluid volume through the outlet port and the second force draws fluid from the vacuum space through the inlet port and into the closed-off fluid volume to create a vacuum condition in the vacuum space.

14. A prosthetic device for attachment to a lower extremity comprising:
at least one pressure element;
a pistonless vacuum pump comprising an elastically deformable material and a wall forming a closed-off fluid volume adjacent the at least one pressure element, the elastically deformable material being positioned within the closed-off fluid volume, the wall being flexible or having flexible portions so that upon application of a first force exerted by the at least one pressure element the wall flexes, the elastically deformable material compresses, and a volume of the closed-off fluid volume reduces, wherein expansion of the elastically deformable material creates a second force directed to move away the pressure element and create a volume increase in the closed-off fluid volume;
an outlet port in fluid communication with the closed-off fluid volume, which evacuates fluid from the closed-off fluid volume upon the application of the first force; and
an inlet port in fluid communication with the closed-off fluid volume and a vacuum space of the prosthetic device, which draws fluid into the closed-off fluid volume upon application of the second force to create a vacuum condition in the vacuum space.

15. The prosthetic device component as claimed in claim 14, wherein the at least one pressure element comprises two spaced rigid walls, and wherein the elastically deformable material is arranged in a space between the two rigid walls.

16. The prosthetic device component as claimed in claim 15, wherein the space between the rigid walls is reduced upon application of the first force.

17. The prosthetic device component as claimed in claim 14, wherein the elastically deformable material comprises a material which is capable of throughflow of a fluid and is arranged within the closed-off fluid volume.

18. The prosthetic device component as claimed in claim 15, wherein the material capable of throughflow comprises an open-pored foam.

19. The prosthetic device component as claimed in claim 17, wherein the material capable of throughflow fills the fluid volume.

20. The prosthetic device component as claimed in claim 14, wherein the elastically deformable material bears against the wall.

21. The prosthetic device component as claimed in claim 20, wherein the wall surrounds the elastically deformable material on all sides.

22. A prosthetic device, comprising:
a pistonless vacuum pump, comprising:
a wall configured to form a closed-off fluid volume, the wall being flexible or having flexible portions movable by means of a first force which produces a volume reduction of the fluid volume and, after a preceding volume reduction, the wall movable by means of a second force which produces a volume increase in the closed-off fluid volume;
an inlet port in fluid communication with the closed-off fluid volume, the inlet port including a non-return valve configured to be connected in fluid communication to a vacuum space of the prosthetic device;
an outlet port in fluid communication with the closed-off fluid volume, the outlet port including a non-return valve configured to exhaust external to the fluid volume; and
an insert positioned within the closed-off fluid volume and comprising an elastically deformable material that is compressed by the first force and whose expansion, after termination of the first force, produces the second force;
wherein the first force exhausts fluid from the closed-off fluid volume through the outlet port and the second force draws fluid from the vacuum space through the inlet port and into the closed-off fluid volume to create a vacuum condition in the vacuum space.
